(19) United States
(12) Patent Application Publication
(10) Pub. No.: US 2022/0177946 A1
(43) Pub. Date: Jun. 9, 2022

(54) PET FOOD RECOMMENDING DEVICE AND PET FOOD RECOMMENDING METHOD, SUPPLEMENT RECOMMENDING DEVICE AND SUPPLEMENT RECOMMENDING METHOD, AND INTESTINAL AGE CALCULATION FORMULA DETERMINING METHOD AND INTESTINAL AGE CALCULATION METHOD

(71) Applicant: DENTSU GROUP INC., Tokyo (JP)

(72) Inventors: Kazuhiko IKEDA, Tokyo (JP); Yoko KODAIRA, Tokyo (JP); Norifumi WATANABE, Tokyo (JP); Izumi HAGINO, Tokyo (JP); Yu SAWAI, Tokyo (JP); Aya TAKEDA, Tokyo (JP); Satoshi WATANABE, Tokyo (JP)

(73) Assignee: DENTSU INC., Tokyo (JP)

(21) Appl. No.: 17/604,363

(22) PCT Filed: Apr. 16, 2020
(86) PCT No.: PCT/JP2020/016659

§ 371 Date: Oct. 15, 2021

(30) Foreign Application Priority Data
Apr. 16, 2019 (JP) 2019-077644

(51) Int. Cl. C12Q 1/06; G01N 33/483

(52) U.S. Cl. CPC C12Q 1/06; A61B 10/0038; G01N 33/4833

(57) ABSTRACT

A pet food recommending device and a pet food recommending method for recommending a food suitable for a pet are provided.

A pet food recommending device is provided, the device including a recommending means that recommends a food suitable for a pet, based on a result of a stool inspection of the pet and attribute information on the pet.

### TECHNICAL FIELD

The present invention relates to a pet food recommending device and a pet food recommending method for recommending a food suitable for a pet, to a supplement recommending device and a supplement recommending method for recommending a supplement suitable for a person, and to an intestinal age calculation formula determining method and an intestinal age calculation method.

### BACKGROUND ART

Today, various pet foods have been proposed (see, for example, Patent Literature 1). However, it is not easy to make a pet food suitable in common for all types of pets. Similarly, various human supplements have also been proposed, and it is not easy to make supplements suitable in common for all types of persons.

Patent Literature 2 discloses a technique for recommending a supplement (functional material) to a person, i.e., recommendation subject. However, the technique of Patent Literature 2 uses questionnaires to users as its key method, and the users' answers to the questionnaires depend heavily on the users' subjectivity. For this reason, an appropriate supplement is not always recommended.

### CITATION LIST

### Patent Literature

### Patent Literature 1: Japanese Unexamined Patent Application Publication No. 2017-2053

### Patent Literature 2: Japanese Patent No. 6245487

### SUMMARY OF INVENTION

### Technical Problem

An object of the present invention is to provide a pet food recommending device and a pet food recommending method for recommending a food suitable for a pet, and a supplement recommending device and a supplement recommending method for recommending a supplement suitable for a person. Another object of the present invention is to provide an intestinal age calculation method and an intestinal age calculation formula determining method for estimating an intestinal age from a bacteria distribution.

### Solution to Problem

According to one aspect of the present invention, a pet food recommending device is provided, the device including a recommending means that recommends a food suitable for a pet, based on a result of stool inspection of the pet and attribute information on the pet.

The result of stool inspection may include information on an intestinal flora of the pet, and the recommending means may recommend the food suitable for the pet, based on information on the intestinal flora and attribute information on the pet.

According to one aspect of the present invention, a pet food recommending device is provided, the device including a recommending means that recommends a food suitable for the pet, based on attribute information on the pet and information on a food ingested by the pet.

The recommending means may estimate information on an intestinal flora of the pet from attribute information on the pet and information on a food ingested by the pet and may recommend a food suitable for the pet, based on the information on the intestinal flora and the attribute information on the pet.

The recommending means may classify the pet into one of a plurality of predetermined groups according to information on the intestinal flora, and a recommended food may be set in advance for each of the plurality of groups.

The recommending means may classify the pet with reference to a diversity of intestinal bacteria possessed by the pet, the number of lactic acid bacteria, the number of butyric acid bacteria, and a level of an FB ratio, based on information on the intestinal flora.

The reference may depend on the attribute information on the pet.

The food may include a base food depending on the attribute information of the pet, and effective bacteria depending on information on the intestinal flora.

The effective bacteria may include bacteria of which the pet is short, based on information on the intestinal flora.

The recommending means may calculate an intestinal age of the pet, based on information on the intestinal flora.

According to one aspect of the present invention, a pet food recommending method is provided, the method including a step of recommending a food suitable for a pet, based on a result of stool inspection of the pet and attribute information on the pet.

According to one aspect of the present invention, a pet food recommending method is provided, the method including a step of recommending a food suitable for the pet, based on attribute information on the pet and information on a food ingested by the pet.

According to one aspect of the present invention, a supplement recommending device is provided, the device including a recommending means that classifies a person into one of a plurality of predetermined groups, based on information on an intestinal flora acquired from a result of stool inspection of the person, and that recommends a supplement suitable for the person, based on a group into which the person is classified.

Information on the intestinal flora may include quantitative data on species and the number of bacteria living in a person's intestines.

The recommending means may classify of the person according to whether species of intestinal bacteria possessed by the person exceed a first reference value, whether lactic acid bacteria exceed a second reference value, whether butyric acid bacteria exceed a third reference value, and whether an FB ratio exceeds a fourth reference value, based on information on the intestinal flora.

The recommending means may recommend a supplement suitable for the person by additionally taking into consideration attribute information on the person.

According to one aspect of the present invention, a supplement recommending method is provided, the method including: inspecting a person's stool to acquire information on an intestinal flora; classifying the person into one of a plurality of predetermined groups, based on the acquired information on the intestinal flora; and recommending a supplement suitable for the person, based on a group into which the person is classified.

According to one aspect of the present invention, an intestinal age calculation formula determining method is provided, the method including: acquiring bacterial flora data on a healthy individual; selecting a bacteria group highly correlated with an actual age, based on a distribution amount of each bacteria group; extracting a principal component by performing dimension compression using the distribution amount of the selected bacteria group as an argument; creating, by regression analysis, a first equation for calculating an intermediate intestinal age, based on the principal component obtained by the dimension compression and on the distribution amount of the selected bacteria group, the first equation including a constant for predicting the actual age from the calculated intermediate intestinal age; and creating, by regression analysis, a second equation for predicting the actual age from the intermediate intestinal age calculated from the created first equation, the second equation including a constant for reducing an error included in a prediction model based on the first equation and the second equation.

According to one aspect of the present invention, an intestinal age calculation method of calculating an intestinal age by applying the first equation and the second equation to a distribution amount of the selected bacteria group is provided.

### Advantageous Effects of Invention

Using attribute information allows recommendation of a fool suitable for a pet or a person. In addition, an intestinal age can be estimated from a distribution of bacteria.

### BRIEF DESCRIPTION OF DRAWINGS

FIG. 1 is a block diagram showing a schematic configuration of a pet food recommending system according to a first embodiment.

FIG. 2 depicts an example of a web page screen for entering attribute information on a pet.

FIG. 3 is a diagram for specifically explaining group classification performed by a recommending means 3.

FIG. 4A is a diagram showing an example of a recommendation screen for a pet classified as a Young-F type.

FIG. 4B is a diagram showing an example of a recommendation screen for a pet classified as a Young-LC type.

FIG. 4C is a diagram showing an example of a recommendation screen for a pet classified as a Young-N type.

FIG. 4D is a diagram showing an example of a recommendation screen for a pet classified as a Young-C type.

FIG. 4E is a diagram showing an example of a recommendation screen for a pet classified as the Young-N type.

FIG. 5 is a food production process chart.

FIG. 6 is a block diagram showing a schematic configuration of a pet food recommending system according to a second embodiment.

FIG. 7 is a block diagram showing a schematic configuration of a pet food recommending system according to a third embodiment.

### DESCRIPTION OF EMBODIMENTS

Embodiments according to the present invention will hereinafter be described specifically with reference to the drawings. In the following description, dogs are taken as an example of a pet, but the description applies also to other pets, such as cats and birds.

### First Embodiment

A first embodiment is summarized as a system that inspects a stool of a pet and that recommends a pet food, based on a result of the inspection and attribute information on the pet.

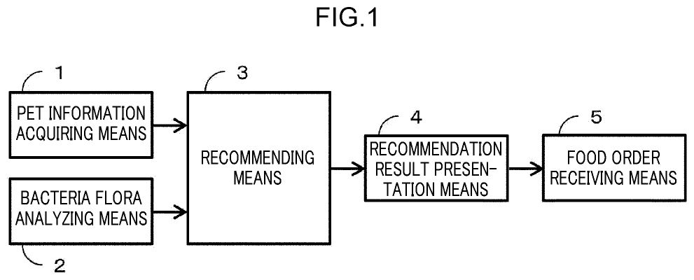

FIG. 1 is a block diagram showing a schematic configuration of a pet food recommending system according to the first embodiment. The pet food recommending system includes a pet information acquiring means 1, a bacterial flora analyzing means 2, a recommending means 3, a recommendation result presentation means 4, and a food order receiving means 5. These means may be included in one system or may be distributed among a plurality of systems. For example, the pet information acquiring means 1, the recommendation result presentation means 4, and the food order receiving means 5 are run on a web server. The bacterial flora analyzing means 2 is provided as a means run by a given inspection institution. The recommending means 3 is run on a server used by an administrator of the present system (obviously, this server may be integrated with the above web server).

The pet information acquiring means 1 acquires attribute information on a pet. More specifically, the pet information acquiring means 1 acquires attribute information on the pet that is manually entered by the owner of the pet in a prescribed form on a website. It is preferable that the attribute information on the pet mentioned here include information of a dog species, weight, age, sex, and a living environment (keeping the pet indoors or outdoors). The attribute information, however, may include some of these pieces of information or other pieces of information, and, as long as it is related to pet food, is not subjected to particular limitations.

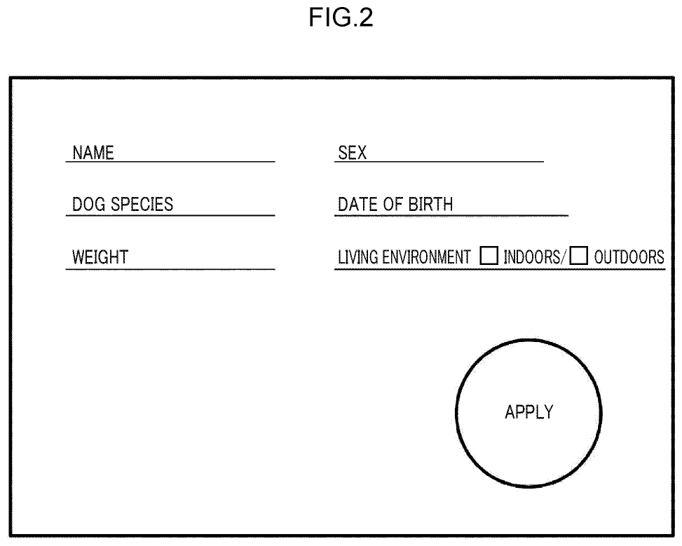

In a specific example, the pet information acquiring means 1 puts a web page shown in FIG. 2 on the display of the owner's terminal device (not illustrated). The pet information acquiring means 1 then receives attribute information entered by the owner, such as the name and dog species of the pet. Subsequently, when an “Apply” button is selected, the entered attribute information is transmitted to the recommending means 3. In addition, a stool inspection kit is delivered to the owner.

The bacterial flora analyzing means 2 shown in FIG. 1 performs a stool inspection of the pet. More specifically, the owner receives the stool inspection kit, collects a stool of the pet, and sends the collected stool to the system. The bacterial flora analyzing means 2 inspects and analyzes the stool to obtain information on an intestinal flora and the like as an inspection result. The intestinal flora refers to the ecology of bacteria living in the intestines of pets. The information on the intestinal flora may include, but is not limited to, for example, the diversity of intestinal bacteria possessed by pets, the number of lactic acid bacteria, the number of butyric acid bacteria (either or both of the genus Faecalibacterium and the genus Clostridium), and an FB ratio which represents a ratio between Fermicutes and Bacteroides and indicates a tendency to get fat. For example, the information may include one or more of the phylum Firmicutes, the phylum Bacteroides, the phylum Proteobacteria, the phylum Actinomycetes, the phylum Fusobacteria, the genus Faecalibacterium, the genus Clostridium, the genus Lactobacillus, the genus Bifidobacterium, the genus Osilospira, the genus Brautia, the genus SMB 53, the genus Fusobacterium, the genus Prevotella, the genus Luminococcus, the genus Thulicobacter, the genus Streptococcus, the genus 02d06, the genus Stetterra, the genus Rosebria, the genus Dorea, and the genus eubacteria.

According to the present embodiment, information on the intestinal flora, which includes such objective and quantitative data as the species and number of bacteria living in the intestines, can be acquired from the result of a stool inspection. This makes it possible to provide an appropriate supplement.

The recommending means 3 recommends a food suitable for the pet, based on the result of stool inspection of the pet and attribute information on the pet. In the present embodiment, it is assumed that the result of stool inspection include information on the intestinal flora. The food to be recommended is not limited to one type, and a plurality of types of foods different in taste may be recommended. Specific recommending methods and recommended foods will be described later.

The recommendation result presentation means 4 presents information on a recommended food to the owner. In a specific example, the recommendation result presentation means 4 posts the information on the recommended food and, preferably, the inspection result of the pet, on a web page accessible to the owner. The recommendation result presentation means 4 may send a medical certificate showing the information on the recommended food and the inspection result, to the owner.

The food order receiving means 5 receives an order for the recommended food, which has been presented to the owner, from the owner. In a specific example, the food order receiving means 5 places an icon for purchasing the recommended food on the web page where the recommended food is displayed. In response to selection of the icon, the food order receiving means 5 carries out necessary settlement transactions. As a result, the recommended food is delivered to the owner. It is better that, with the weight of the pet being taking into consideration, the volume of the food be determined to be, for example, a volume that the pet would consume in a certain period (one month or the like).

A specific recommending method by the recommending means 3 will then be described as an exemplary example.

The recommending means 3 classifies the pet into one of a plurality of predetermined groups according to information on the intestinal flora. A recommended food is set in advance for each group. In other words, the recommending means 3 classifies the pet into a specific group, and recommends a food set for the group.

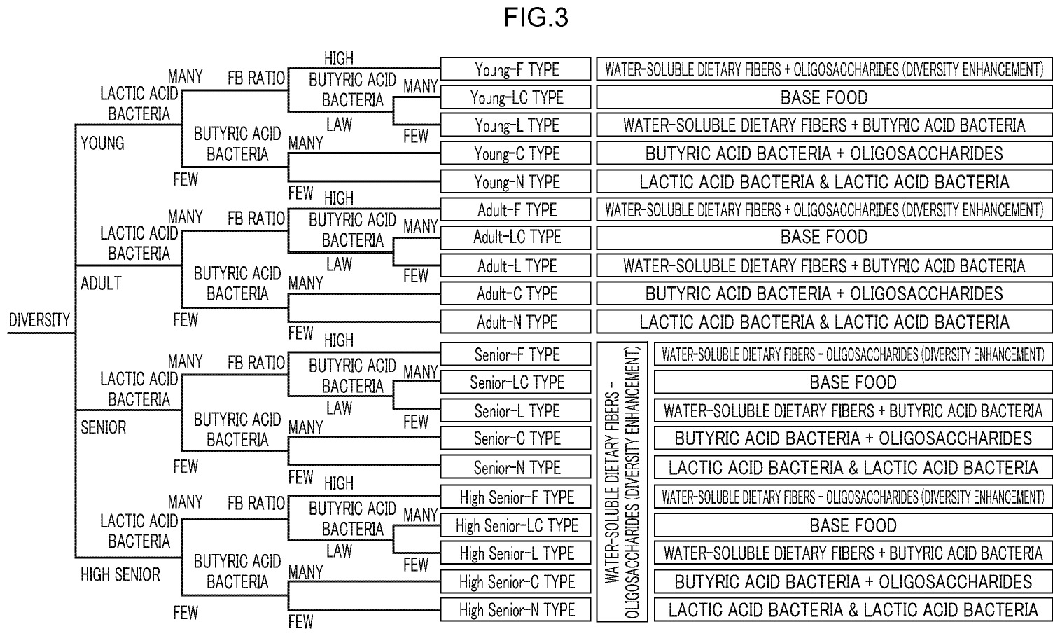

FIG. 3 is a diagram for specifically explaining group classification performed by the recommending means 3. The recommending means 3 performs group classification based on the diversity of intestinal bacteria, and group classification based on lactic acid bacteria, butyric acid bacteria, and the FB ratio.

First, the recommending means 3 classifies pets into a plurality of groups (four groups shown in FIG. 3), based on the diversity of intestinal bacteria included in information on the intestinal flora. In many cases, the younger pets are, the more diversified their intestinal bacteria are. For convenience, therefore, group names “Young, Adult, Senior, and High Senior” are given to groups in their order of richness in the diversity. The diversity of intestinal bacteria is determined, for example, depending on whether species of bacteria living in the intestine exceeds a reference value.

Subsequently, the recommending means 3 further classifies each group classified according to the diversity, into one of two groups according to the number of lactic acid bacteria. In a specific example, this classification can be made depending on whether the number of lactic acid bacteria is larger than a reference value.

The recommending means 3 then further classifies a group with a large number of lactic acid bacteria into one of two groups, according to the level of the FB ratio. In a specific example, this classification can be made depending on whether the FB ratio is higher than a reference value. A group with a high FB ratio is defined as an F type. A group with a low FB ratio, on the other hand, is further classified into two groups according to the number of butyric acid bacteria. In a specific example, this classification can be made depending on whether the number of butyric acid bacteria is larger than a reference value. A group with a large number of butyric acid bacteria is defined as an LC type, and a group with a small number of butyric acid bacteria is defined as a C type.

In addition, the recommending means 3 further classifies a group with a small number of lactic acid bacteria into one of two groups, according to the number of butyric acid bacteria. In a specific example, this classification can be made depending on whether the number of butyric acid bacteria is larger than a reference value. A group with a large number of butyric acid bacteria is defined as a C type, and a group with a small number of butyric acid bacteria is defined as an N type.

It can be said that the F type is a group having many lactic acid bacteria but showing a high FB ratio, that the LC type is a group having many lactic acid bacteria as well as many butyric acid bacteria, that the L type is a group having many lactic acid bacteria but having few butyric acid bacteria, that the C type is a group having many butyric acid bacteria but having few lactic acid bacteria, and that the N type is a group having few lactic acid bacteria as well as few butyric acid bacteria.

As described above, the pet is classified into one of 4 groups, according to the diversity of intestinal bacteria, and is further classified into one of 5 groups, based on lactic acid bacteria/butyric acid bacteria and the FB ratio. Hence the pet is classified into one of 20 groups, which is the total of classification groups. For example, when a pet is classified into the Young category by diversity-based classification and into the F type by classification based on the number of lactic acid bacteria/butyric acid bacteria and on the FB ratio, a group into which the pet is finally classified is a Young-F type.

It is desirable that each reference value used in group classification be a value corresponding to attribute information on the pet, such as the dog species, age, and weight of the pet. Classification made according to the number of lactic acid bacteria and butyric acid bacteria includes classification made according to the presence or absence of those bacteria.

A recommended food can be provided as a food composed of a base food and several % of a necessary effective bacteria food mixed into the base food. The base food may be independent of information on the intestinal flora, but should desirably be one prepared in accordance with attribute information on the pet. Specifically, the base food should preferably be of a type suitable for the actual age of the pet and have a weight suitable for the weight of the pet. The effective bacteria, on the other hand, correspond to a group into which the pet is classified based on information on the intestinal flora, and should preferably contain bacteria the pet is short of and a substance that activates (supports) such bacteria.

A pet belonging to the F type group has rich lactic acid bacteria, which is preferable, but has a high FB ratio, which gives the pet a tendency to get fat. For the F type group, therefore, in addition to the base food, a food containing water-soluble dietary fibers and oligosaccharides for diversity enhancement is recommended as an effective bacteria food.

The LC type represents an ideal bacterial flora containing many lactic acid bacteria as well as many butyric acid bacteria. The LC type group, therefore, does not need an effective bacteria hood, and the base food is recommended for this group.

The L type represents a bacterial flora containing many lactic acid bacteria but few butyric acid bacteria. For the L type group, therefore, in addition to the base food, a food containing butyric acid bacteria and water-soluble dietary fibers that activate the butyric acid bacteria is recommended as an effective bacteria food.

Type C represents a bacterial flora containing many butyric acid bacteria but few lactic acid bacteria. For the type C group, therefore, in addition to the base food, a food containing lactic acid bacteria and oligosaccharides that activate the lactic acid bacteria is recommended as an effective bacteria food.

The N type represents a bacterial flora containing few butyric acid bacteria as well as few butyric acid bacteria. For the N type group, therefore, in addition to the base food, a food containing lactic acid bacteria and butyric acid bacteria is recommended as an effective bacteria food.

Because the Senior and High Senior groups have intestinal bacteria that are low in diversity, an effective bacteria food containing more water-soluble dietary fibers and oligosaccharides may be recommended not only for the F type but also for the LC type, the L type, the C type, and the N type of those Senor and High Senor groups.

Comparing the Young-F type with the Adult-F type reveals that an effective bacteria food containing water-soluble dietary fibers and oligosaccharides is recommended in common for both Young-F type with the Adult-F type. However, because the diversity of intestinal bacteria of the Adult-F type is lower, an effective bacteria food containing more water-soluble dietary fibers and oligosaccharides is recommended for the Adult-F type. In this manner, groups belonging to the same F type category have different effective bacteria foods recommended thereto. Water-soluble dietary fibers and oligosaccharides making up the effective bacterial foods are greater in amount in the order of the Young-F type, the Adult-F type, the Senior-F type, and the High Senior-F type. This applies also to groups belonging to the LC type, L type, C type, and N type categories.

It is also desirable that a food recommended for a pet belonging to a specific type group be a food prepared in accordance with attribute information on the pet.

For example, Chihuahua, one dog species, needs more calcium (because of its thin bones), oligosaccharides, dietary fibers (because of its sensitive gastrointestinal tracts), and omega-3 fatty acids (for maintaining healthy joints) than other dog species. It is desirable, for this reason, that, for Chihuahua classified into, for example, the Young-F type, a food containing more calcium, oligosaccharides, dietary fibers, and omega-3 fatty acids than a food for other dog species classified into the same Young-F type be recommended.

In another example, dogs at high actual ages consume less energy, and are prone to get constipation due to lack of exercise and reduced intestinal activity. It is therefore desirable that, for example, for an old dog (for example, 8 years old or older) classified into the Young-F type, a small amount of high-calorie food containing more dietary fibers than a food for a young dog (for example, up to 1 year old) or an adult dog (for example, 1 to 8 years old) classified into the same Young-F type be recommended.

In another example, dogs kept indoors tend to get fat. It is therefore desirable that, for example, for an indoor dog classified into the Young-F type, to maintain its muscle mass and increase its basic metabolism, a food higher in protein content and lower in calorie than a food for an outdoor dog classified into the same Young-F type be recommended.

In this manner, the food recommended for the pet is determined. Thus, the recommendation result presentation means 4 puts the following web pages (which will hereinafter be referred to as “recommendation screens”) on display.

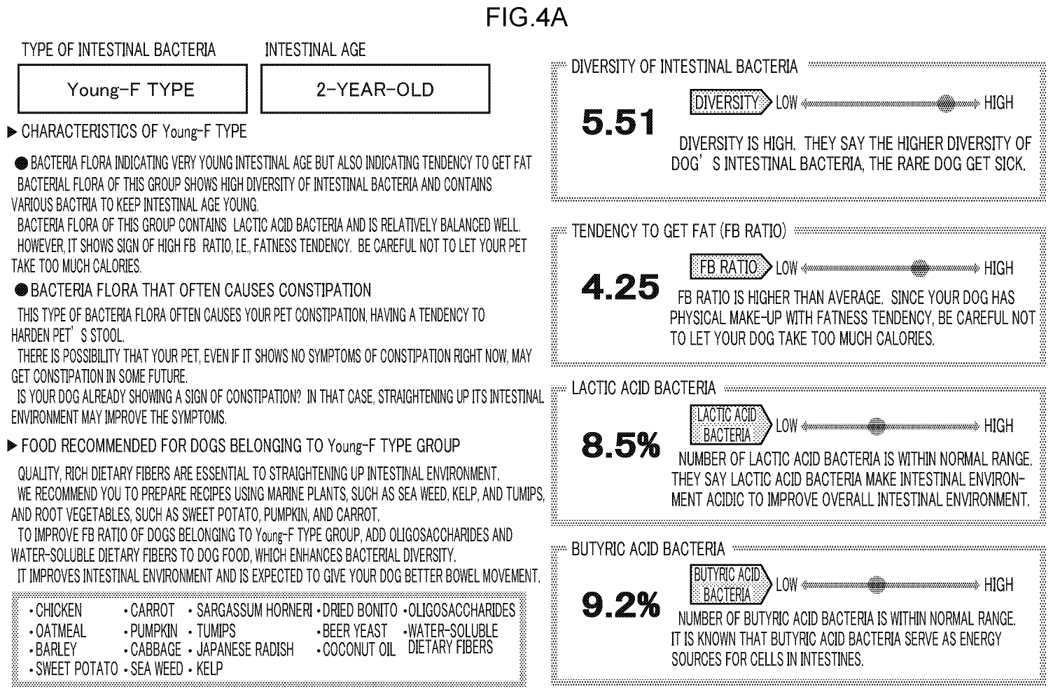

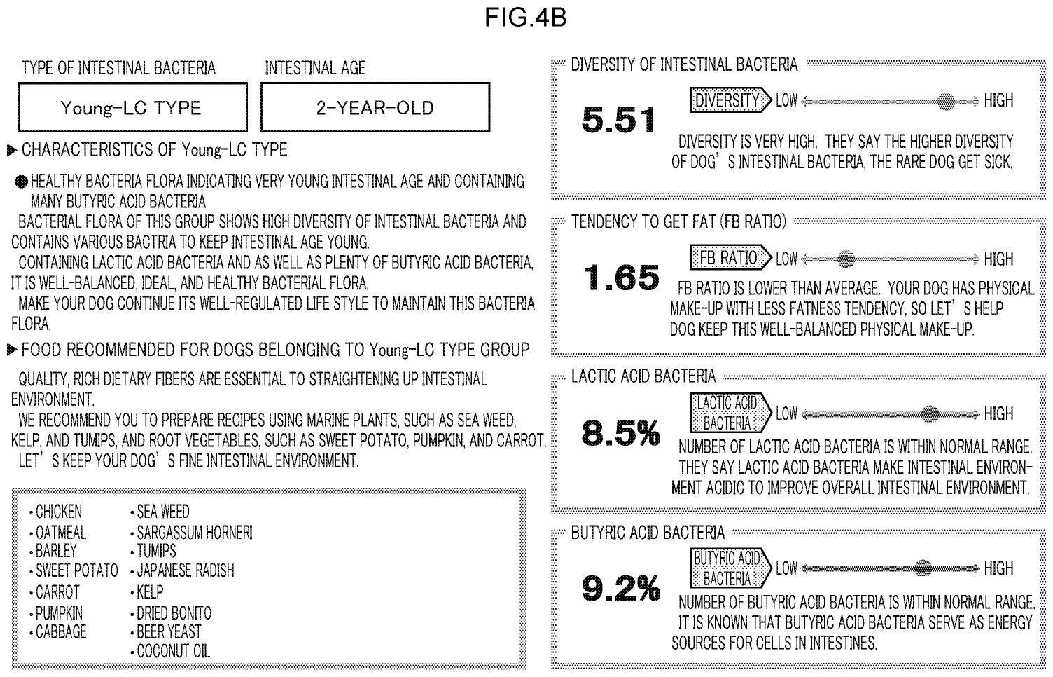

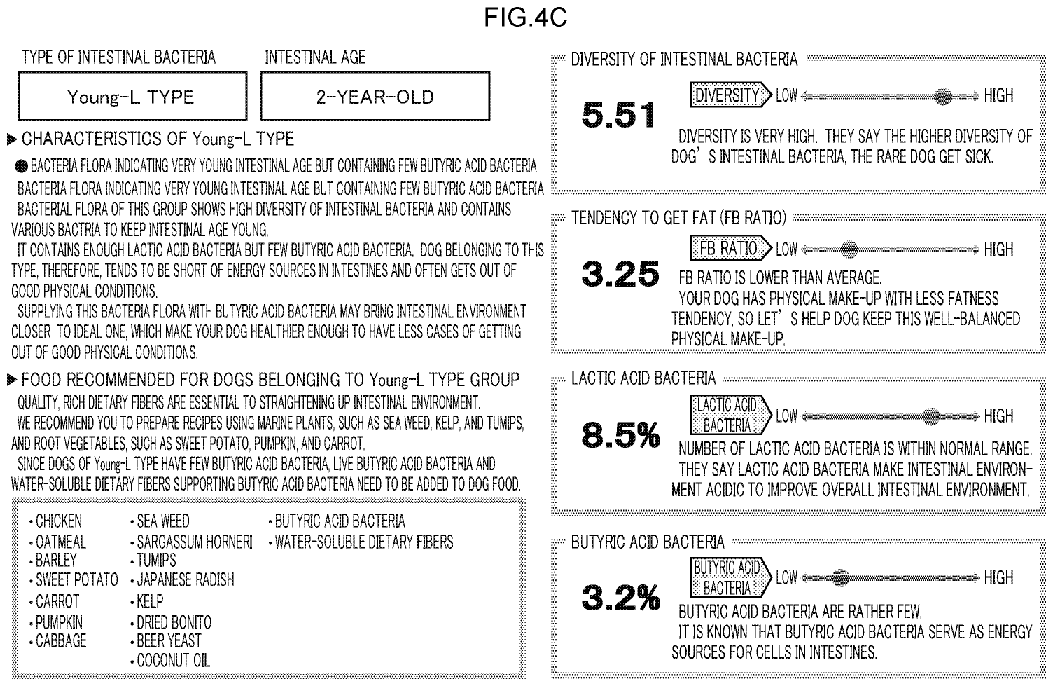

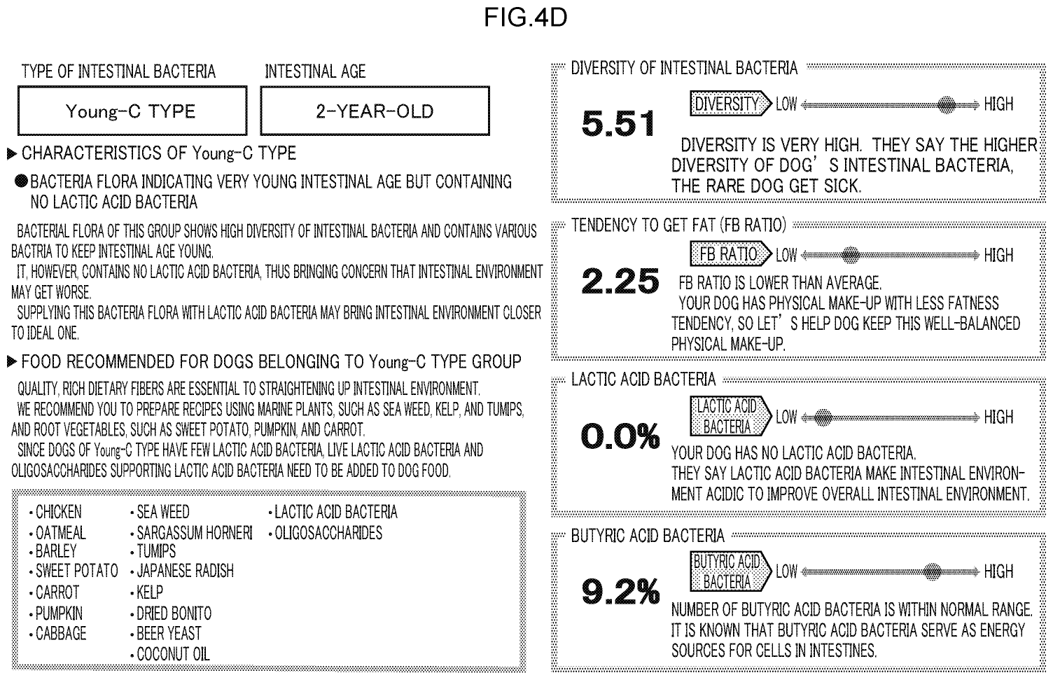

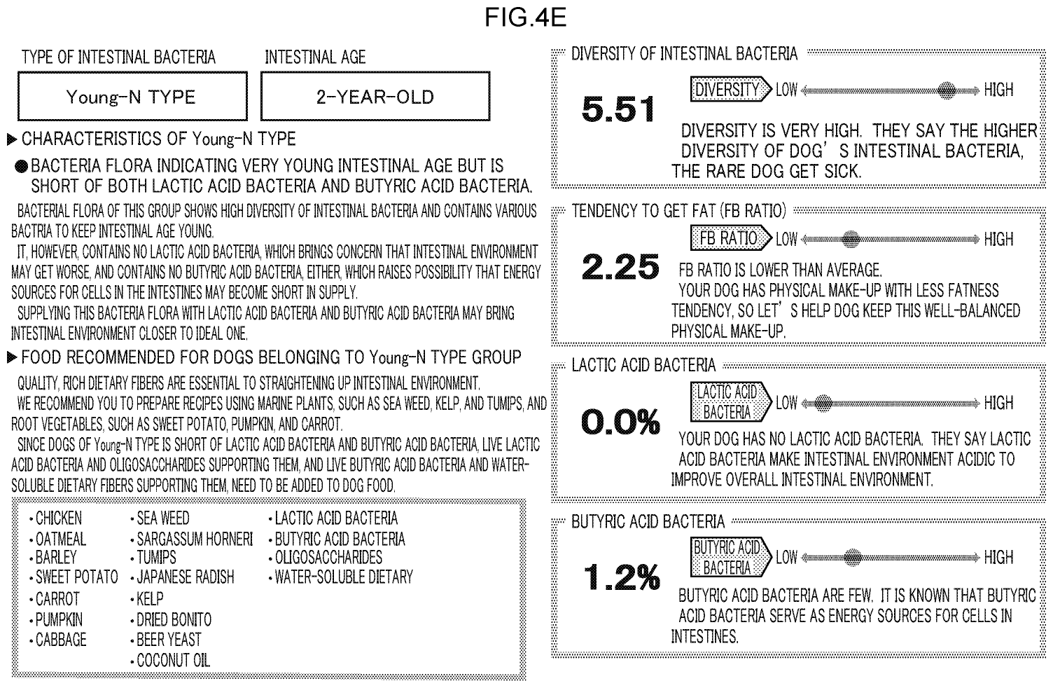

FIGS. 4A to 4E are diagrams showing examples of recommendation screens for pets classified respectively into the Young-F type, the Young-LC type, the Young-N type, the Young-C type, and the Young-N type. As shown in FIGS. 4A to 4E, each recommendation screen includes descriptions of a type in which a pet is classified and the features of the type. The screen also displays specific feeds. It is desirable that the recommendation screen further express the diversity of intestinal bacteria, the FB ratio, lactic acid bacteria, and butyric acid bacteria used for group classification, as numerical values, and display simple comments and cautions.

In addition, the recommendation screen may include an “intestinal age” calculated by the recommending means 3. The intestinal age is a parameter indicating the age of the intestinal flora, and is calculated based on information on the intestinal flora, attribute information, and the like. The intestinal age may be calculated according to the type and sex of the pet. A specific example of an intestinal age calculation method will be described in a fourth embodiment.

As described above, a recommended food is made up of a base food and an effective bacteria food. This makes a food production process efficient.

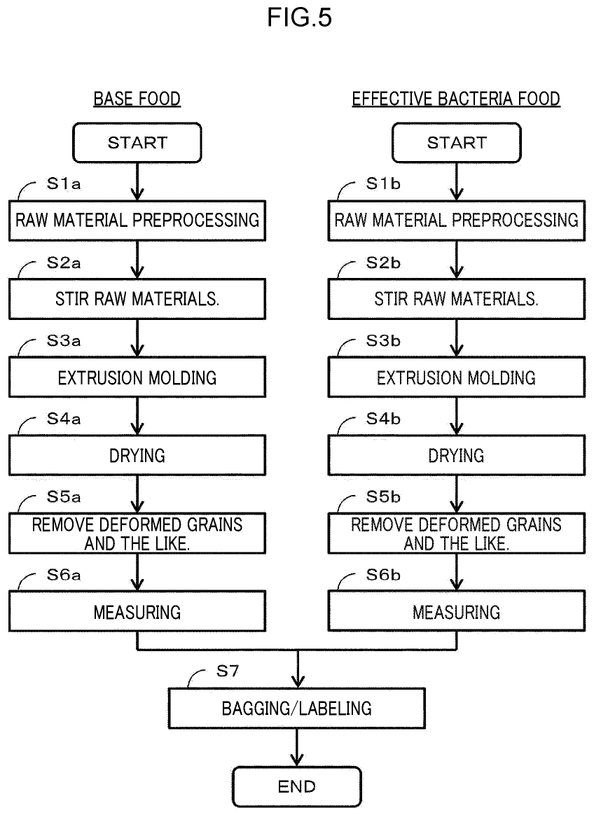

FIG. 5 is a food production process chart. A base food is produced by the following steps. First, raw material preprocessing, such as pulverizing materials, is carried out (step S1a). Subsequently, the raw material is stirred (step S2a), and is extrusion-molded while being heated by an extruder (step S3a). The raw material is then dried under a high temperature (step S4a). Then, deformed grains and the like are removed from the material (step S5a) to complete the base food.

An effective bacteria food is produced by the following steps. First, raw material preprocessing, such as baking, dry grinding, and mincing chicken, is carried out (step S1b). Subsequently, effective bacteria are added to the material, which is stirred (step S2b) and is extrusion-molded by an extruder without being exposed to heat (step S3b). The material is then dried at a temperature low enough to allow the effective bacteria to stay alive (step S4b). Then, deformed grains and the like are removed from the material (step S5b) to complete the base food.

Subsequently, the base food and the effective bacteria food are measured out in desired amount, respectively (steps S6a and 6b), and are put into bags, which are labeled (step S7) to complete the food.

In this manner, according to the first embodiment, a food suitable for the pet can be recommended by taking into consideration information on the intestinal flora (bacterial diversity, FB ratio, butyric acid bacteria, lactic acid bacteria, etc.) and attribute information on the pet (dog species, weight, age, sex, living environment, etc.). Particularly, according to the present embodiment, a stool inspection allows acquisition of quantitative measured values, such as the diversity of intestinal bacteria, the number of individual bacteria groups, such as lactic acid bacteria and butyric acid bacteria, and the FB ratio. As a result, pets can be classified objectively to allow recommendation of an appropriate food.

In the present embodiment, information on the intestinal flora and attribute information on the pet are both used, but only the information on the intestinal flora may be used. Furthermore, a food recommended based on information on the intestinal flora may be corrected by attribute information.

### Second Embodiment

A second embodiment is summarized as a system that dispenses with a stool inspection of a pet carried out in the first embodiment and that uses information on a food ingested by the pet, in place of a stool inspection result.

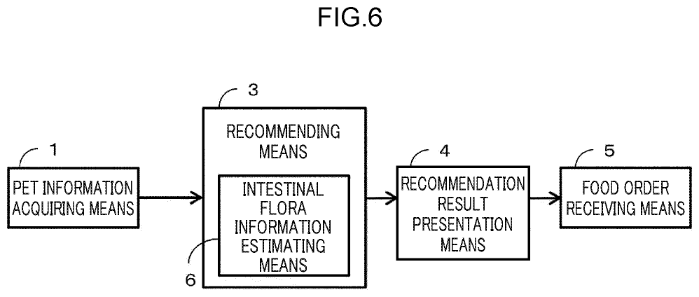

FIG. 6 is a block diagram showing a schematic configuration of a pet food recommending system according to the second embodiment. Hereinafter, differences with the first embodiment will be mainly described.

The pet information acquiring means 1 of this pet food recommending system acquires information on a food ingested by a pet, in addition to attribute information on the pet. In a specific example, the pet information acquiring means 1 displays a web page for inputting a food ingested (in a desirable case, ingested most recently) by the pet, on the owner's terminal device, and receives input from the owner.

Then, the recommending means 3 recommends a food suitable for the pet, based on the attribute information on the pet and the information on the food ingested by the pet. Specifically, the recommending means 3 has an intestinal flora information estimating means 6. The intestinal flora information estimating means 6 estimates information on the intestinal flora of the pet from the attribute information on the pet and the information on the food ingested by the pet. In a specific example, the intestinal flora information estimating means 6 holds a database indicating a relationship between the attribute information on the pet, the information of the food ingested by the pet, and the intestinal flora, and refers to the database, thereby estimating the information on the intestinal flora.

The second embodiment is the same as the first embodiment in other respects, except that estimated intestinal flora information is used in place of intestinal flora information obtained by a bacterial flora analysis.

According to the second embodiment, inspecting a stool of the pet is unnecessary, and therefore a food suitable for the pet can be recommended more easily.

### Third Embodiment

The above first and second embodiments provide systems that recommend a food for a pet. A third embodiment to be described next, in contrast, provides a system that recommends a human food (supplement in most cases).

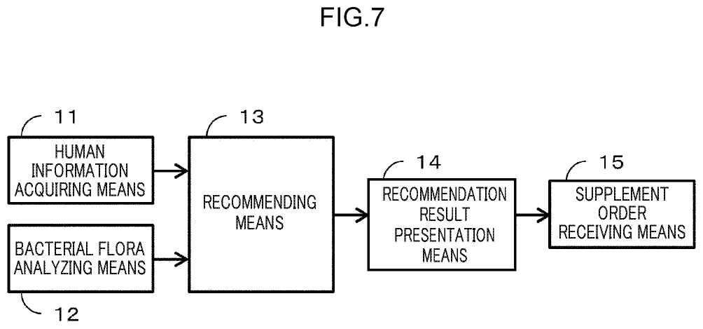

FIG. 7 is a block diagram showing a schematic configuration of a human supplement recommending system according to the third embodiment. The human supplement recommending system includes a human information acquiring means 11, a bacterial flora analyzing means 12, a recommending means 13, a recommendation result presentation means 14, and a supplement order receiving means 15. These means may be included in one system or may be distributed among a plurality of systems. For example, the human information acquiring means 11, the recommendation result presentation means 14, and the supplement order receiving means 15 are run on a web server. The bacterial flora analyzing means 12 is provided as a means run by a given inspection institution. The recommending means 13 is run on a server used by an administrator of the present system (obviously, this server may be integrated with the above web server).

The human information acquiring means 11 acquires attribute information on a person. More specifically, the human information acquiring means 11 acquires attribute information on a person that is manually entered by the person who is a recommendation subject (or by a family member who knows the person well) in a prescribed form on a website. It is preferable that attribute information on the person mentioned here include information of the weight, age, sex, and dietary habits of the person. The attribute information, however, may include some of these pieces of information or other pieces of information, and, as long as it is related to human supplements, is not subjected to particular limitations.

In a specific example, the human information acquiring means 11 puts a web page similar to the web page of FIG. 2 on a display (note that “dog species” and “living environment” are removed from the web page). The human information acquiring means 11 then receives entries of attribute information on the person, i.e., the recommendation subject. Subsequently, when an “Apply” button is selected, the entered attribute information is transmitted to the recommending means 13. In addition, a stool inspection kit is delivered to the person, i.e., the recommendation subject.

The bacterial flora analyzing means 12 performs a stool inspection of the person, i.e., the recommendation subject. More specifically, the person, i.e., the recommendation subject receives the stool inspection kit, collects his or her stool, and sends the collected stool to the system. The bacterial flora analyzing means 12 inspects and analyzes the stool to obtain information on an intestinal flora and the like as an inspection result. The intestinal flora refers to the ecology of bacteria living in the person's intestines. Information on the intestinal flora may include, but is not limited to, for example, the diversity of intestinal bacteria possessed by a person, the number of lactic acid bacteria, the number of butyric acid bacteria (either or both of the genus Faecalibacterium and the genus Clostridium), and an FB ratio which represents a ratio between Fermicutes and Bacteroides and indicates a tendency to get fat. For example, the information may include one or more of the phylum Firmicutes, the phylum Bacteroides, the phylum Proteobacteria, the phylum Actinomycetes, the phylum Fusobacteria, the genus Faecalibacterium, the genus Clostridium, the genus Lactobacillus, the genus Bifidobacterium, the genus Osilospira, the genus Brautia, the genus SMB 53, the genus Fusobacterium, the genus Prevotella, the genus Luminococcus, the genus Thulicobacter, the genus Streptococcus, the genus 02d06, the genus Stetterra, the genus Rosebria, the genus Dorea, and the genus eubacteria.

According to the present embodiment, information on the intestinal flora, which includes such objective and quantitative data as the species and number of bacteria living in the intestines, can be acquired from the result of a stool inspection. This makes it possible to provide an appropriate supplement.

The recommending means 13 recommends a food suitable for the person, based on the result of stool inspection of the person and attribute information on the person. In the present embodiment, it is assumed that the result of stool inspection include information on the intestinal flora. A supplement to be recommended is not limited to one type, and a plurality of types of supplements different in taste may be recommended.

The recommendation result presentation means 14 presents information on a recommended supplement to the person, i.e., the recommendation subject (or to a member of the person's family). In a specific example, the recommendation result presentation means 14 posts the information on the recommended supplement and, preferably, the inspection result of the person, on a web page accessible to the person, i.e., the recommendation subject. The recommendation result presentation means 4 may send a medical certificate showing the information on the recommended supplement and the inspection result, to the person.

The food order receiving means 15 receives an order for the recommended supplement, which has been presented. In a specific example, the supplement order receiving means 15 places an icon for purchasing the recommended supplement on the web page where the recommended supplement is displayed. In response to selection of the icon, the supplement order receiving means 15 carries out necessary settlement transactions. As a result, the recommended supplement is delivered to the person.

It should be noted that a specific recommendation method carried out by the recommending means 13 may be similar to the recommendation method described in the first embodiment. Also in the present embodiment, a stool inspection allows acquisition of quantitative measured values, such as the diversity of intestinal bacteria, the number of individual bacteria groups, such as lactic acid bacteria and butyric acid bacteria, and the FB ratio. As a result, persons can be classified objectively to allow recommendation of an appropriate food.

In addition, similarly to the second embodiment, the stool inspection may be omitted and information on a food ingested by the person may be used, instead. In other words, the human information acquiring means 11 acquires information on a food ingested by the person, in addition to attribute information on the person. In a specific example, a web page for entering a food ingested (in a desirable case, ingested most recently) by the person is displayed on the person's terminal device, where an entry of the food is received on the web page.

Then, the recommending means 13 recommends a supplement suitable for the person, based on attribute information on the person and information on the food ingested by the person. Specifically, the recommending means 13 estimates information on the intestinal flora of the person from the attribute information on the person and the information on the food ingested by the person. In a specific example, the recommending means 13 holds a database indicating a relationship between the attribute information on the person, the information on the food ingested by the person, and the intestinal flora, and refers to the database, thereby estimating the information on the intestinal flora.

The third embodiment is the same as the third embodiment in other respects, except that estimated intestinal flora information is used in place of intestinal flora information obtained by a bacterial flora analysis.

In this manner, according to the third embodiment, a food suitable for a person can be recommended by taking into consideration information on the intestinal flora (bacterial diversity, FB ratio, butyric acid bacteria, lactic acid bacteria, etc.) and attribute information on the person (weight, age, sex, dietary habits, etc.). In the present embodiment, information on the intestinal flora and attribute information on the person are both used, but only the information on the intestinal flora may be used. Furthermore, a food recommended based on information on the intestinal flora may be corrected by attribute information.

### Fourth Embodiment

In a fourth embodiment to be described next, a specific example of the above intestinal age calculation method will be described. An intestinal age is an index numerically expressing a health condition of the intestines with reference to an actual age, using a distribution data on an intestinal bacteria flora. An intestinal age prediction model will be described on the assumption that a distribution amount of intestinal bacteria has a certain tendency of correlation with age and that the intestinal age and the actual age of a healthy individual match up.

In this prediction model, it is assumed that when the actual age is y0, the intestinal age y can be calculated based on the following equations.

y={(y1−y0)+a}/b+y0 (1)

where y1=k1{f(a1,a2 . . . an)}+k2{g(a1,a2 . . . an)} (2)

For convenience, a variable y1 is referred to as “intermediate intestinal age”. Hereinafter, arguments a1 to an, functions f and g, and constants k1, k2, a, and b in equations (1) and (2) will be described in order.

The arguments a1 to an represent distribution amounts and indexes of bacteria having high correlation with the actual age and providing a high possibility of predicting the actual age, and are selected by a statistical method. Specifically, the arguments a1 to an can be selected in the following manner.

First, certain standards are set by referring to medical histories, clinical histories, medication statuses, health conditions, and the like, and bacterial flora data on a healthy individual that meets the standards is collected. Then, a distribution amount of each bacteria group (which is a ratio of amount of each bacteria group to the total amount of bacteria analyzed by a sequencer, and is a continuous value ranging from 0 to 1) is standardized by logit transformation to create a data set for analysis. Subsequently, based on a contribution ratio obtained by a bootstrap method, bacteria groups and indexes that are highly correlated with the actual age are adopted as an intestinal age calculation model.

Analysis by the inventors has found that it is preferable to adopt distributions of such bacteria groups as bifidobacteria (k_Bacteria; p_Actinobacteria; c_Actinobacteria; o_Bifidobacteriales; f_Bifidobacteriaceae; g_Bifidobacterium), lactic acid bacteria (k_Bacteria; p_Firmicutes; c_Bacilli; o_Lactobacillales; f_Lactobacillaceae; g_Lactobacillus), and butyric acid bacteria (k_Bacteria; p_Firmicutes; c_Clostridia; o_Clostridiales; f_Clostridiaceae; g_Clostridium), as arguments.

In another case, other indexes different from bacteria-based indexes (diversity indexes or distribution amounts of multiple bacteria species included in supplied food) may also be adopted as arguments.

In the above-described manner, the arguments a1 to an are determined (“n” denotes the total of the number of bacteria groups adopted and the number of other indexes). It should be noted that bacteria groups different from the bacteria groups mentioned above and other indexes may be adopted as arguments.

Next, a principal component analysis is carried out on the arguments a1 to an to visualize a relationship between the arguments and extract principal components through dimension compression.

Analysis by the inventors has revealed that a first principal component and a second principal component may be adopted according to the interpretability of each of these principal components. In the above equation (2), a function for calculating the first principal component is f, and a function for calculating the second principal component is g. It should be noted that a third principal component and other principal components to follow may be adopted.

After the first principal component and second principal component are calculated, the first principal component and second principal component are adjusted, based on an average distribution amount of each bacteria group, and the functions f and g in the above equation (2) are determined.

In addition, constants k1 and k2 for predicting the actual age y0 from the intermediate intestinal age y1 in the above equation (2) are determined by regression analysis.

Further, constants a and b for predicting the actual age y0 from the intermediate intestinal age y1 are determined by regression analysis. It can be said that the constants a and b are constants that are adjusted to reduce an error included in the prediction model.

In the above manner, the arguments and constants for calculating the intestinal age are determined, and the above equations (1) and (2) are applied to the intestinal flora (a1 to an) to calculate the intestinal age y.

It is assumed that the intestinal age calculation method is applied mainly to pets. However, the calculation method may also be applied to persons.

The above embodiments have been described for the purpose of enabling an individual having ordinary knowledge of the technical field to which the present invention belongs to implement the present invention. Various modifications of the above embodiment can obviously be made by those skilled in the art, and the technical concept of the present invention can be applied to other embodiments. The present invention is, therefore, not limited to the described embodiments, and its scope should be the widest one in accordance with the technical concept defined by the claims.

### REFERENCE SIGNS LIST

1 pet information acquiring means
2 bacterial flora analyzing means
3 recommending means
4 recommendation result presentation means
5 food order receiving means
6 Intestinal flora information estimating means
11 human information acquiring means
12 bacterial flora analyzing means
13 recommending means
14 recommendation result presentation means
15 supplement order receiving means

1.-19. (canceled)

20. A pet food recommending apparatus comprising a circuitry configured to:
recommend a food suitable for a pet based on intestinal flora information of the pet and attribute information of the pet, wherein the intestinal flora information is based on a result of a stool inspection of the pet and relates to species and number of bacterial included in an intestine of the pet, and the attribute information includes at least one of species, weight, age, sex and living environment of the pet,
wherein the circuitry is further configured to classify the pet into one of a plurality of groups using a variation diversity of intestinal bacteria possessed by the pet based on the intestinal flora information, as a reference,
a food to be recommended is set in advance for each of the plurality of groups,
the recommended food comprises:
effective bacteria of which the pet is short based on the intestinal flora information, and
a base food depending on the attribute information of the pet.

21. The apparatus according to claim 20, wherein the circuitry is configured to classify the pet using, based on the intestinal flora information, (i) the variation diversity of intestinal bacteria possessed by the pet, and (ii) number of lactic acid bacteria or number of butyric acid bacteria, as the reference.

22. The apparatus according to claim 20, wherein the circuitry is configured to classify the pet using, based on the intestinal flora information, (i) the variation diversity of intestinal bacteria possessed by the pet, (ii) number of lactic acid bacteria, and (iii) number of butyric acid bacteria, as the reference.

23. The apparatus according to claim 20, wherein the reference depends on the attribute information of the pet.

24. The apparatus according to claim 20, wherein the food further comprises a substance that activates the effective bacteria of which the pet is short.

25. A method performed by a circuitry, the method comprising:
recommending a food suitable for a pet based on intestinal flora information based on a result of a stool inspection of the pet and attribute information, wherein the intestinal flora information relates to species and number of bacterial included in an intestine of the pet, and the attribute information includes at least one of species, weight, age, sex and living environment of the pet,
wherein the recommending further comprises classifying the pet into one of a plurality of groups using a variation diversity of intestinal bacteria possessed by the pet based on the intestinal flora information, as a reference,
a food to be recommended is set in advance for each of the plurality of groups,
the food comprises:
effective bacteria of which the pet is short based on the intestinal flora information, and
a base food depending on the attribute information of the pet.

26. An apparatus comprising a circuitry configured to:
obtain attribute information of a pet and food information on a food ingested by the pet;
estimate intestinal flora information from the obtained attribute information and the food information by referring to a database in which information on intestinal flora is associated with attribute information and food information; and
recommend a food suitable for the pet based on the estimated intestinal flora information and the attribute information,
wherein the attribute information includes at least one of species, weight, age, sex and living environment of the pet,
the food information is information on the food ingested by the pet and registered in the database,
the recommended food comprises:
effective bacteria of which the pet is short based on the intestinal flora information, and
a base food depending on the attribute information of the pet.

27. The apparatus according to claim 26, wherein the circuitry is configured to classify the pet into one of a plurality of groups using a variation diversity of intestinal bacteria possessed by the pet based on the intestinal flora information, as a reference,
wherein a food to be recommended is set in advance for each of the plurality of groups.

28. The apparatus according to claim 26, wherein the reference depends on the attribute information of the pet.

29. The apparatus according to claim 26, wherein the food further comprises a substance that activates the effective bacteria of which the pet is short.

30. A pet food recommending method performed by a circuitry, the method comprises:
obtaining attribute information of a pet and food information on a food ingested by the pet;
estimating intestinal flora information from the obtained attribute information and the food information by referring to a database in which information on intestinal flora is associated with attribute information and food information; and
recommending a food suitable for the pet based on the estimated intestinal flora information and the attribute information,
wherein the attribute information includes at least one of species, weight, age, sex and living environment of the pet,
the food information is information on the food ingested by the pet and registered in the database,
the recommended food comprises:
effective bacteria of which the pet is short based on the intestinal flora information, and
a base food depending on the attribute information of the pet.
